(19) United States
(12) Patent Application Publication
(10) Pub. No.: US 2022/0409515 A1
(43) Pub. Date: Dec. 29, 2022

(54) RINSE FREE PERSONAL CARE COMPOSITIONS

(71) Applicant: The Procter & Gamble Company, Cincinnati, OH (US)

(72) Inventors: Yonas Gizaw, West Chester, OH (US); Roy Jerome Harrington, Liberty Township, OH (US); Iris Bi Liu, Mason, OH (US); Nathaniel Okposio, Fairfield, OH (US); Matthew Scott Wagner, Cincinnati, OH (US); Steven Hardy Page, Lawrenceburg, IN (US)

(21) Appl. No.: 17/840,659

(22) Filed: Jun. 15, 2022

Related U.S. Application Data
(60) Provisional application No. 63/211,010, filed on Jun. 16, 2021.

(51) Int. Cl. A61K 8/81; A61Q 5/02; A61K 8/73; A61K 8/39; A61K 8/34; A61K 8/365; A61K 8/86; A61K 8/49

(52) U.S. Cl. CPC A61K 8/8182; A61Q 5/02; A61K 8/731; A61K 8/39; A61K 8/345; A61K 8/365; A61K 8/86; A61K 8/4973

(57) ABSTRACT

Personal care compositions comprising a polymer and a trisolvent system, and the use of the compositions in removing oily soils (such as grease or sebum).

### FIELD OF THE INVENTION

The present disclosure relates to personal care compositions comprising a polymer and a trisolvent system, and the use of the compositions in removing oily soils (such as grease or sebum). More particularly the present disclosure relates to personal care compositions for out of shower gentle removing of sebum and spreading of sebum on hair without the use of surfactants that strip natural lubricating oils from scalp and hair.

### BACKGROUND OF THE INVENTION

In general, cleaning hair requires a shampoo composition that contains a surfactant as a main ingredient and further contains additives such as a preservative, a fragrance, etc. and water. It is common practice to use shampoo compositions based essentially on standard surfactants, such as anionic, nonionic and/or amphoteric type surfactants, but more particularly of anionic type, to clean and wash hair. Anionic surfactants are useful in shampoo compositions; however, they can be problematic in that they can facilitate hair damage or cause irritation and promote color fading from dyed hair due to excessive cleansing ability. To compensate for the powerful cleaning ability of surfactants, consumers generally apply conditioners to replace the stripped oil from their hair. Furthermore, the use of shampoo to clean hair requires excessive rinsing to remove the remaining surfactant from hair. Rinsing consumes more hot water demanding high level of energy consumption and increased carbon footprints. Saving water becomes critical in regions of the world with low water and energy availability. After washing with shampoo, conditioning and rinsing, consumers generally air and blow dry hair to style to the desired end point; with the drying process consuming energy, time, and further damaging hair.

Thus, what is needed is a composition that provides sufficient cleaning while avoiding hair damage and loss of hair shine, while reducing water and energy usage. The current composition using an inventive polymer and tri-solvent system removes sebum with minimal water or energy usage, while leaving the hair undamaged with a natural looking shine.

### SUMMARY OF THE INVENTION

A personal care composition comprising, about 0.01% to about 1.0% polymer, by weight of the composition, wherein the polymer is cationic either through a quaternary amine or pH dependent chargeable mono-, di-, or tri-alkyl amine; wherein the cationic polymer has a ratio of cationic or pH dependent chargeable monomers to noncationic or non-chargeable monomers of from about 1:99 to about 20:80; wherein the polymer has a molecular weight of from about 10,000 to about 10,000,000. And from about 1.0% to about 30%, by weight of said composition, of a triple solvent system with surface tension of less than about 45 mN/m; comprising a first solvent, a second solvent, and a third solvent, wherein each of said first solvent, said second solvent, and said third solvent are different; wherein HLB value of the solvents is from about 6.25 to about 8.5; wherein the composition has a viscosity of about 1.0 cps to about 10 cps; wherein the composition is substantially surfactant free; wherein the composition removes at least about 25% or more artificial sebum measured by the Extraction and UV-VIS analysis methods, and spreads at least about 20%, as determined by root % spread plus length % spread, of the non-removed artificial sebum from the roots to the remaining hair length as measured by the SSRT method.

A personal care composition comprising from about 1% to about 20%, by weight of said composition, of a solvent system comprising a first solvent, a second solvent, and a third solvent, wherein each of said first solvent, said second solvent, and said third solvent are different; wherein said first solvent and said second solvent are each independently at least one of propylene glycol n-butyl ether, tripropylene glycol methyl ether, dipropylene glycol n-propyl ether, propylene glycol propyl ether, dipropylene glycol n-butyl ether, tripropylene glycol n-butyl ether, diethylene glycol n-butyl ether, diethylene glycol hexyl ether, diethylene glycol n-butyl ether acetate, ethylene glycol hexyl ether, triethylene glycol methyl ether, triethylene glycol ethyl ether, triethylene glycol n-butyl ether, 2,2,4-trimethyl-1,3-pentanediol monoisobutyrate, trimethylnonanol, propylene glycol diacetate, and dipropylene glycol methyl ether; and wherein said third solvent is at least one of propylene glycol n-butyl ether, tripropylene glycol methyl ether, dipropylene glycol n-propyl ether, dipropylene glycol n-butyl ether, propylene glycol propyl ether, tripropylene glycol n-butyl ether, diethylene glycol n-butyl ether, diethylene glycol hexyl ether, diethylene glycol n-butyl ether acetate, ethylene glycol hexyl ether, triethylene glycol methyl ether, triethylene glycol ethyl ether, triethylene glycol n-butyl ether, 2,2,4-trimethyl-1,3-pentanediol monoisobutyrate, trimethylnonanol, propylene glycol diacetate, dipropylene glycol methyl ether, ethanol, isopropanol, isopropyl myristate, propylene glycol, dipropylene glycol, hexylene glycol, ethoxy diglycol, or 1,2-hexanediol; and from about 0.01% to about 1.0%, by weight of said composition, of a polymer, wherein said polymer is at least one of:

(i) polymers having the following formula:

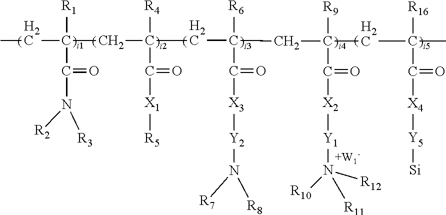

wherein
R₁, R₂, R₃, R₄, R₆, R₇, R₈, R₉, and R₁₆ are each independently selected from H or C₁-C₆ alkyl;
X₁, X₂, X₃ and X₄ are each independently selected from O or NH;
R₅ is C₈-C₃₀ n- or iso-alkyl;
Y₁, Y₂, and Y₅ are each independently selected from —(CH₂)ₘ—, wherein m is 1-30;
R₁₀, R₁₁, and R₁₂ are each independently selected from C₁-C₆ alkyl; preferably R₁₀, R₁₁, and R₁₂ are each independently selected from methyl, ethyl and propyl;
W₁⁻ is a counter ion; preferably W₁⁻ is selected from Cl⁻, Br⁻, I⁻, HSO₄⁻, CH₃SO₄⁻, C₂H₅SO₄⁻, or OH⁻;
Si is a silicone or derivative thereof, preferably Si is at least one of a polydimethylsiloxane, an aminosilicone, a cationic silicone, a silicone polyether, a cyclic silicone, a fluorinated silicone and mixtures thereof; preferably Si is polydimethylsiloxane; preferably Si is a silicone or derivative thereof having a molecular weight of from about 250 to about 40,000, preferably from about 500 to about 20,000, more preferably from about 1,000 to about 10,000 Da;
i₁ is an integer selected such that the monomer units constitute from about 30% to about 99.8% by weight of Polymer (i);
i₂ is an integer selected such that the monomer units constitute from about 0.1% to 50% by weight of Polymer (i);
i₃ is an integer selected such that the monomer units constitute from about 0.1% to 50% by weight of Polymer (i);
i₄ is an integer selected such that the monomer units constitute from 0% to 30% by weight of Polymer (i); and
i₅ is an integer selected such that the monomer units constitute from 0% to 20% by weight of Polymer (i);
(ii) polymers having the following formula:

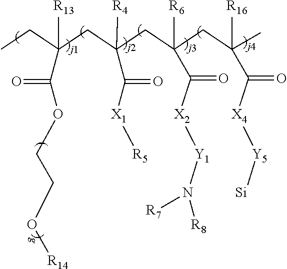

wherein
R₁₃, R₁₄, R₄, R₆, R₇, R₈ and R₁₆ are each independently selected from H or C₁-C₆ alkyl;
g is from 0 to 100, preferably g is 1;
X₁, X₂, and X₄ are each independently selected from O or NH;
R₅ is C₈-C₃₀ n- or iso-alkyl;
Y₁ and Y₅ are each independently selected from —(CH₂)ₘ—, wherein m is 1-30;
Si is a silicone or derivative thereof; preferably Si is at least one of a polydimethylsiloxane, an aminosilicone, a cationic silicone, a silicone polyether, a cyclic silicone, a fluorinated silicone and mixtures thereof; preferably Si is polydimethylsiloxane; preferably Si is a silicone or derivative thereof having a molecular weight of from about 250 to about 40,000, preferably from about 500 to about 20,000, more preferably from about 1,000 to about 10,000 Da;
j₁ is an integer selected such that the monomer units constitute from about 50% to 100% by weight of Polymer (ii);
j₂ is an integer selected such that the monomer units constitute from 0% to 50% by weight of Polymer (ii); and
j3 is an integer selected such that the monomer units constitute from 0% to 50% by weight of Polymer (ii); and
j4 is an integer selected such that the monomer units constitute from 0% to 50% by weight of Polymer (ii);
(iii) polymers having the following formula:

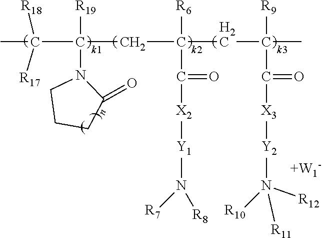

wherein
R₁₇, R₁₈, R₁₉, R₆, R₇, R₈, and R₉ are each independently selected from H or C₁-C₆ alkyl;
n is 0-4, preferably n is 1-4;
X₂ and X₃ are each independently selected from O or NH;
Y₁ and Y₂ are each independently selected from —(CH₂)ₘ—, wherein m is 1-30;
R₁₀, R₁₁, and R₁₂ are each independently selected from C1-C6 alkyl; preferably R10, R11, and R₁₂ are each independently selected from methyl, ethyl and propyl;
W₁— is a counter ion; preferably W₁— is selected from Cl—, Br—, I—, HSO₄⁻, CH3SO₄⁻, C2H5SO₄⁻, or OH—;
k₁ is an integer selected such that the monomer units constitute from about 50% to 100% by weight of Polymer (iii);
k₂ is an integer selected such that the monomer units constitute from 0% to about 50% by weight of Polymer (iii); and
k₃ is an integer selected such that the monomer units constitute from 0% to about 50% by weight of Polymer (iii); or
(iv)
polymers having a randomly substituted polysaccharide backbone comprising unsubstituted and substituted glucopyranose monomers and having a general structure according to Formula I:

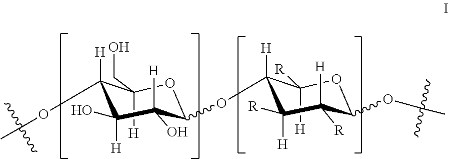

wherein each substituted glucopyranose monomer independently comprises from 1 to 3 R substituents, which may be the same or different on each substituted glucopyranose monomer, and
each R substituent is independently a substituent selected from the group consisting of hydroxyl, hydroxymethyl, R¹, R², R³ and a polysaccharide branch having a general structure according to Formula I; or a substituent selected from the group consisting of hydroxyl, hydroxymethyl, R¹, R², and a polysaccharide branch having a general structure according to Formula I, provided that at least one R substituent comprises at least one R¹ and at least one R²,
each R¹ is independently, the same or different, a first substituent group having a degree of substitution ranging from 0.01 to 0.2 and a structure according to Formula II:

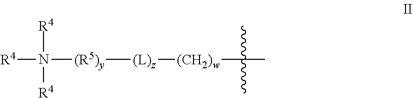

each R⁴ is a substituent selected from the group consisting of H; CH₃; linear or branched, saturated or unsaturated C₂-C₁₈ alkyl, provided that the sum of number of carbons of at least two of the R⁴ groups does not exceed 24, R⁵ is a linear or branched, saturated or unsaturated C₂-C₁₈ alkanediyl or a linear or branched, saturated or unsaturated secondary hydroxy(C₂-C₁₈)alkanediyl, L is a linking group selected from the group consisting of —O—, —C(O)O—, —NR⁹—, —C(O)NR⁹—, and —NR⁹C(O)NR⁹—, and R⁹ is H or C₁-C₆ alkyl, w has a value of 0 or 1, y has a value of 0 or 1, and z has a value of 0 or 1,
each R₂ is independently, the same or different, a second substituent group having a degree of substitution ranging from 0.001 to 0.5 and a structure according to Formula III:

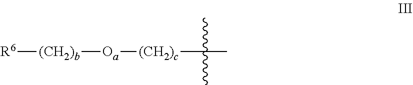

R⁶ is a substituent selected from the group consisting of carboxylate, carboxymethyl, succinate, sulfate, sulfonate, arylsulfonate, phosphate, phosphonate, dicarboxylate, and polycarboxylate, a has a value of 0 or 1, b is an integer from 0 to 18, and c has a value of 0 or 1,
each R³ is independently, the same or different, a third substituent group having a degree of substitution of 0 or ranging from 0.001 to 1.0, and having a structure according to Formula IV:

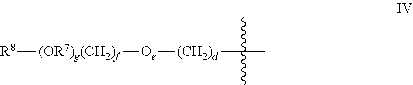

d has a value of 0 or 1, e has a value of 0 or 1, f is an integer from 0 to 8, g is an integer from 0 to 50, each R⁷ is the group ethanediyl, 1,2-propanediyl, 1,2-butanediyl, or mixtures thereof, and R⁸ is an end group selected from the group consisting of hydrogen, C₁-C₂₀ alkyl, hydroxy, —OR¹ and —OR², and
the polymer has a weight average molecular weight ranging from 10,000 to 10,000,000 Daltons or from about 200,000 to 3,000,000; or
   (v) mixtures thereof;
   from 0% to about 0.05%, by weight of said cleaning composition, of surfactant; and at least about 80%, by weight of said cleaning composition, of water.

The present invention further relates to a consumer product comprising the personal care composition contained in a bottle in fluid communication with a sprayer assembly.

The present invention further relates to a consumer product comprising a nonwoven substrate impregnated with the personal care composition.

The present invention further relates to a consumer product comprising a sprayer device with the personal care composition.

The present invention further relates to methods of using the personal care composition for cleaning a variety of surfaces.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 Shows an illustration of a TA. XT. Plus Texture Analyzer with Attachment.

FIG. 2 Shows an illustration of a Compression Gig.

FIG. 3 Shows an illustration of an attachment of a Hair Tress to a Compression Gig Attachment.

FIG. 4 Illustrates Hair tress attachment onto TA instrument for cleaning experiments.

FIG. 5 Illustrates a Paraffin Wax Calibration Curve.

### DETAILED DESCRIPTION OF THE INVENTION

The present invention provides a personal care composition, for both humans and animals, having substantially no surfactant. The present disclosure relates to personal care compositions comprising a polymer and a trisolvent system, and the use of the compositions in removing oily soils (such as grease or sebum). The combination of the solvent system and polymer results in a cleaning composition which is both low sudsing and improves the loosening and removal of oily soils. It is believed that the combination of solvent and polymer also improves the distribution and deposition of the polymer onto the surface and improves the wetting and emulsification of the oily soil off the treated surface. Further in embodiments viscosity of the personal care composition is important for application, to allow for proper application (i.e. spraying) and for adherence to the hair and sebum, and can range from about 1.0 cps to about 10 cps. The personal care composition may comprise the trisolvent system in embodiments from about 1% to about 30%, about 1% to about 25%, about 1% to about 20%, or from about 10% to about 20%.

The present invention provides consistent, reproducible sebum/artificial soil removal to provide a user with a feeling of cleanliness and also has the ability to spread the sebum allowing the hair to feel smooth and manageable. The ability of the present invention to both remove and spread sebum contrasts with current shampoos that remove most or all of the sebum leaving the hair dry and brittle, such that the hair requires further treatment in the form of a conditioner. In embodiments a personal care composition of the present invention can remove at least about 15%, 25%, 35% or 45% more artificial sebum as measured by the Extraction and UV-VIS analysis methods, as discussed below. In embodiments a personal care composition of the present invention can spread at least about 20%, 35%, 50%, 75% or 85% as determined by root % spread plus length % spread, of the non-removed artificial sebum from the roots to the remaining hair length as measured by SSRT methods discussed below.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity, unless otherwise designated. All numeric ranges are inclusive of narrower ranges; delineated upper and lower range limits are combinable to create further ranges not explicitly delineated.

All numerical parameters are to be understood as being prefaced and modified in all instances by the term “about” unless otherwise indicated. At the very least, and not as an attempt to limit the application of the doctrine of equivalents to the scope of the claims, each numerical parameter described herein should be construed in light of the number of reported significant digits and by applying ordinary rounding techniques.

Also, any numerical range recited herein is intended to include all sub-ranges of the same numerical precision subsumed within the recited range. For example, a range of “1.0 to 10.0” is intended to include all subranges including and between the recited minimum value of 1.0 and the recited maximum value of 10.0—that is, having a minimum value equal to or greater than 1.0 and a maximum value equal to or less than 10.0—such as, for example, 1.4 to 7.6 or 8.1 to 9.7. Any maximum numerical limitation in any numerical range recited in this specification is intended to include all lower numerical limitations subsumed therein; and any minimum numerical limitation in any numerical range recited in this specification is intended to include all higher numerical limitations subsumed therein. Accordingly, Applicant reserves the right to amend this specification, including the claims, to expressly recite any sub-range subsumed within the ranges expressly recited herein. All such ranges are intended to be inherently described in this specification such that an amendment expressly reciting any such sub-range would comply with the requirements of 35 U.S.C. § 112(a).

Where amount ranges are given, these are to be understood as being the total amount of said ingredient in the composition, or where more than one species fall within the scope of the ingredient definition, the total amount of all ingredients fitting that definition, in the composition.

The amount of each particular ingredient or mixtures thereof described hereinafter can account for up to 100% (or 100%) of the total amount of the ingredient(s) in the personal care composition.

“Apply” or “application,” as used in reference to a composition, means to apply or spread the compositions of the present invention onto a body surface, such as hair, and scalp

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Soluble” means at least about 0.1 g of solute dissolves in 100 ml of solvent, at 25° C. and 1 atm of pressure.

As used herein, the term “solvent” does not include water.

The term “substantially surfactant free” or “substantially free of surfactant” as used herein means less than about 1%, or less than about 0.8%, or less than about 0.5%, or less than about 0.3%, less than about 0.01%, less than about 0.001%, or about 0%, of surfactant by total weight of the composition.

“Hair,” as used herein, means mammalian hair including scalp hair, facial hair and body hair, particularly hair on the human head and scalp.

“Cosmetically acceptable,” as used herein, means that the compositions, formulations, or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

As used herein, the term “fluid” includes liquids and gels.

As used herein, the term “Room Temperature” or “RT”, refers to an average ambient temperature of between about 20° C. to about 25° C.

As used herein, the articles including “a” and “an” when used in a claim, are understood to mean one or more of what is claimed or described.

As used herein, the word “or” when used as a connector of two or more elements is meant to include the elements individually and in combination; for example X or Y, means X or Y or both.

As used herein, “comprising” means that other steps and other ingredients which do not affect the end result can be added. This term encompasses the terms “consisting of” and “consisting essentially of”.

As used herein, “mixtures” is meant to include a simple combination of materials and any compounds that may result from their combination.

As used herein, a “surfactant” is a compound that lowers the surface tension between two liquids, between a gas and a liquid, or between a liquid and a solid. Surfactants adsorb to the air/water interface and reduce the surface tension of water. A surfactant can be defined as a chemical meeting all five of the following criteria: 1) used in detergent, has surface-active properties, and consists of hydrophilic and hydrophobic groups, 2) capable of reducing the surface tension of water to below 45 mN/m, 3) of forming emulsions and/or microemulsions and/or micelles, 4) adsorption at water/solid interface, and 5) forming spreading or adsorption monolayers at the water-air interface.

By “personal care composition” is meant a product, which in the ordinary course of usage is applied to or contacted with a body, scalp and hair surface to provide a beneficial effect. Examples of personal care compositions include a product applied to a human body for improving appearance, cleansing, and odor control or general aesthetics.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair or nails. Examples of personal care compositions include a product applied to a human body for improving appearance, cleansing, and odor control or general aesthetics. Non-limiting examples of personal care compositions include after shave gels and creams, pre-shave preparations, shaving gels, creams, or foams, moisturizers and lotions; leave-on skin lotions and creams, shampoos, body washes, body rubs, hair conditioners, hair dyeing and bleaching compositions, mousses, masks, shower gels, bar soaps, antiperspirants, deodorants, or hair cleaning compositions for animals, such as dogs and cats.

In certain embodiments the personal care compositions may comprise about 0.01% or more of cationic polymer (or mixtures of cationic polymers), for example from about 0.01% to about 1% or from about 0.02% to about 2% cationic polymer, by weight of the composition. In a hair cleaning context, the composition is used for removing sebum during washing. The cationic polymer can have ratio of cationic or pH dependent chargeable monomers to noncationic or non-chargeable monomers from 1:99 to 20:80 and a molecular weight of from about 10,000 to about 10,000,000 or from about 200,000 to about 3,000,000. The cationic polymer can be either a synthetic copolymer or modified naturally derived polymers.

It is further believed charge density and molecular weight together provide both the conditioning/hydrating feel/wet slickness of the formula and the desired sebum cleaning.

### Test Methods

### Polymers Structure Analysis Using NMR Method

The NMR Content Method is used to determine the ratio on a molar basis of functional groups with different NMR signals.

In this method, proton NMR spectroscopy is used to analyze a sample of soluble material in deuterated water, and peaks of different ¹H-NMR domains are integrated, and ratioed to determine the monomer ratio of protons of the functional groups or different ¹H-NMR domains, respectively.

A flowable solution less than 10% by wt sample dissolved in D₂O and pH adjusted to 6-7 is prepared. The solution is transferred to a glass grade NMR tube and placed in the sample holder (bore) of a proton NMR instrument. An example of a suitable instrument is a Bruker NMR device with 600 MHZ field strength (Bruker Co., Billerica, Mass.). Instruments of other makes and other field strengths, even including instruments operating as low as 300 MHz, can successfully be used to perform this method. A quantitative proton sequence is used to acquire the data. One of skill in the art will be familiar with appropriate choice of other specific data collection parameters. Appropriate parameters used with the exemplary 600-MHz Bruker instrument above are: acquisition time (FID length) of 4.1 s, relaxation time of 60 s, 90-degree pulse widths, spectral width of 20 ppm, 64 k points in the FID, and 16 repetition scans used. In the Fourier transform step, exponential apodization is used with 0.3-Hz line broadening, and the spectrum is phased into absorption. A spline baseline correction is used to ensure flat baseline on either side of peaks to be integrated.

The following peak domains are typically used for the content determination and integrated:
1) The CH region assigned to polyvinylpyrrolidone (PVP unit) 4.2-3.4 ppm (Assignment following e.g. K. Dutta, A. S. Brar, Journal of Polymer Science Part A Polymer Chemistry 37 (21), 3922-3928 (1999)).
2) The two CH3 groups bound to Nitrogen assigned to the N, N di-methyl group 3.0-2.7 ppm (CH3 groups were assigned using HSQC EDITED experiments).

Di-methyl amino group to Polyvinylpyrrolidone unit is calculated as follows:

Soft Quat %=(integral Soft Quat/6)/((integral Soft Quat/6)+integral PVP)*100

PVP %=(integral PVP)/((integral Soft Quat/6)+integral PVP)*100

### Polymer Molecular Weight Measurement

Gel Permeation Chromatography with Multi-Angle Light Scattering and Refractive Index Detection (GPC-MALS/RI) for Polymer Molecular Weight Distribution Measurement

Gel Permeation Chromatography (GPC) with Multi-Angle Light Scattering (MALS) and Refractive Index (RI) Detection (GPC-MALS/RI) permits the measurement of absolute MW of a polymer without the need for column calibration methods or standards. The GPC system allows molecules to be separated as a function of their molecular size. MALS and RI allow information to be obtained on the number average (Mn) and weight average (Mw) molecular weight. The Mw distribution of water-soluble polymers is typically measured by using a Liquid Chromatography system (e.g., Agilent 1260 Infinity pump system with OpenLab Chemstation software, Agilent Technology, Santa Clara, Calif., USA) and a column set (e.g., Waters ultrahydrogel guard column, 6 mm ID×40 mm length, two ultrahydrogel linear columns, 7.8 mm ID×300 mm length, Waters Corporation of Milford, Mass., USA) which is operated at 40° C. The mobile phase is 0.1M sodium nitrate in water containing 0.02% sodium azide and is pumped at a flow rate of ˜1 mL/min, isocratically. A multiangle light scattering (MALS) detector DAWN® and a differential refractive index (RI) detector (Wyatt Technology of Santa Barbara, Calif., USA) controlled by Wyatt Astra® software are used A sample is typically prepared by dissolving polymer materials in the mobile phase at ˜1 mg per ml and by mixing the solution for overnight hydration at room temperature. The sample is then filtered through a 0.8 μm Versapor membrane filter (PALL, Life Sciences, NY, USA) into the LC autosampler vial using a 3-ml syringe before the GPC analysis. A dn/dc (differential change of refractive index with concentration) value is measured on the polymer materials of interest and used for the number average and weight average molecular weights determination by the Astra detector software.

### Soil Spreading and Removal Test (SSRT) Method

The objective for this method is to determine the amount sebum transferred from hair to a substrate by applying constant force and sliding the substrate across the hair length.

In place of natural sebum, for reproducibility, a sebum like substitute (artificial sebum) is used, as described below.

The method described herein lists the procedures for programming an instrument(s) for soil application and removal measurements and describes the treatment and the process in which products are prepared for measurement on hair or hair like substrates.

### Equipment and Materials

| | |
|---|---|
| Instron Force and Friction Analyzer, Model X | Instron Co., Norwood, MA |
| Texture Analyzer | Stable Microsystems, Surry England |
| TA. XT. Plus Texture Analyser with Attachment | Texture Technologies Co., Hamilton, MA |
| (Shown in FIG. 1, Reference No. 10) | |
| UV-Vis Spectrophotometer (VWR UV3100-PC) | VWR International Co., Radner, PA |
| ID (EQU-00000338) SN (ULC 1801021) | |
| Centrifuge Tube (50 ml) | |
| Iso-Propyl Alcohol (CAS-NO-67-63-0) Lot # | EMD Millipore Corporation, Burlington, MA |
| Mo50864-007 (>99.5%) | |
| Acrodisc Syringe Filters (Lot# 4855) Acrodisc | Pall Corporation, New York, NY |
| 25 mm w/0.2 um supor | |
| CEL-GRO Tissue Culture Rotator (SN- | Thermo Scientific, Waltham, MA |
| C1662100926357) Volt 120 | |
| Synthetic PVC Hair Tress 4 g/8″ | International Hair Importers, Glendale, NY |
| General Population Hair 4 g/8″ SN-GP-FN-3R | |
| Artificial sebum (Paraffin Oil Lot# M18021WX 75%, | |
| and the wax 25% Kester Wax K-24 Batch# 1901520) | |

Measurements were conducted under controlled temperature and humidity. (Humidity 55%, and Temperature 22° C.)

### Artificial Hair Tresses Cleaning Method

The artificial hair tresses need to be thoroughly cleaned prior to using for the evaluations.

### Method/Instrument Controls

Instrument Control
   (a) Hair Type: synthetic/artificial hair 1^{st} use, 4 gm 8″ combing tress configuration
   (b) Water temperature: 37.8° C. (100° F.)
   (c) Water flow: 5.7 lpm (1.5 gpm)
   (d) Water hardness: 9 gr
   (e) Water Pressure: 47 psi
   (f) Product amount: 0.1 g Pantene Pro-V Sheer volume shampoo per gram of hair
   (g) Cycle Definition: Lather shampoo and Rinse+

### 6.7 Standard Wash Method.

(a) Adjust water temperature to 37° C.±2° C.; Pressure 324 kPa; water flow rate to ˜5.7 lpm (liters per minute).
(b) Use 0.1 cc global wash (Pantene Pro-V Sheer volume) per 1-gram hair.
(c) Place synthetic hair switch(es) into switch clamp.
(d) Wet/rinse the synthetic hair thoroughly with water.
(e) Apply appropriate amount of global wash (Pantene Pro-v Sheer volume) to front of switch(es); hand wash for 30 seconds.
(f) Rinse for 30 seconds with water.
(g) Turn synthetic switch(es) around to have back of switch facing forward.
(h) Apply appropriate amount of global wash (Pantene Pro-v Sheer volume) to front of switch(es); hand wash for 30 seconds, then rinse for 30 seconds.
(i) Comb hair switch with large teeth 5 times, then with fine teeth of comb 3 times.
(j) Rinse hair switch(es) with water for 2 minutes.
(k) Air dry hair switches in a controlled temperature and humidity room (50% RH/70 F) for 24 hours prior to use.

### Artificial Sebum Preparation

Materials:
Paraffin Oil [Mineral Oil] (CAS #8042-47-5), EMD Millipore, Burlington, Mass., Saybolt Viscosity 340-365 at 37° C., Part #PX0045-3, Lot #551135182) Kester Wax K-24, Koster Keunen, Product #358B, Lot #1901520

Preparation Method:
a) In a weighing vessel, measure 25.00 grams of Kester Wax K-24.
b) Transfer to glass jar.
c) Add 75.00 grams of Paraffin oil [Mineral Oil] to the jar containing the previously weighed wax.
d) Place the lid on the jar and sonicate in a 75 Watt Sonication bath for 30 minutes to disperse the wax in the oil.

### Artificial Sebum Viscosity

Instrument: TA Instruments DHR-2 Discovery Hybrid Rheometer with a Peltier temperature control system. (TA Instruments, New Castle, Del.)

Initial Instrument Parameters:
Initial Temperature (Fixed Plate): 25° C.
Measurement Fixture: 60 mm diameter Titanium parallel plate
Mapping Protocol: instrument and the rotational mapping is performed (3 iterations, precision)
Gap Referencing: Zero to plate surface
Sample load: Excess
Trimming Gap: 1050 microns
The sample is trimmed using a flat plastic blade (eg a plastic microscope slide or a flat ended plastic spatula)
Measurement Gap: gap is lowered to 1000 microns.

Reference Sample (Validating Sample): A viscosity standard (S600 from Cannon) is run at 25° C. to verify that the instrument is working properly.

Condition No. 1 (Stress Sweep and Temperature Ramp)
1) The viscosity of the artificial sebum is measured at 25° C. and 37° C.
2) Using a stress sweep experiment with the following conditions.
3) The temperature is set to either 25° C. or 37° C.
4) Inherit set point is set to Off and the soak time is set to 120 seconds.
5) The stress is varied from 0.1 Pa to 10 Pa with 10 points per decade and logarithmic spacing.
6) Steady state sensing is set to Off.
7) The equilibration time is 25 seconds per point and the data are averaged over the last 5 seconds.
   a. Two replicates are run at 25° C., using a fresh sample for each run
   b. One replicate is run at 37° C., using a fresh sample for each run.
8) The data is fit to a Newtonian model to obtain the viscosity for each run.

Condition No. 2
The viscosity of the artificial sebum is also measured using a Flow Temperature Ramp.
1) The start temperature is set to 25° C.
2) The soak time is set to 0 seconds and wait for temperature is set to On.
3) The ramp rate is 1.0° C./min and the end temperature is set to 40° C.
4) The Shear Rate is set to 10 s⁻¹ with a sampling interval of 1.0 second/point.

Physical Properties—Artificial Sebum: Viscosity
At 25° C., viscosity is 53.0 cP+/−1.2 cP
At 37° C., viscosity is 31.6 cP+/−0.1 cP
   Three measurements of the artificial sebum were taken for each temperature and the average value of the three results is reported as the viscosity of the artificial sebum at a given temperature.

### Texture Analyzer (Ta) Functionality Method

### Texture Analyzer (TA) Equipment Start-Up/Set-Up:

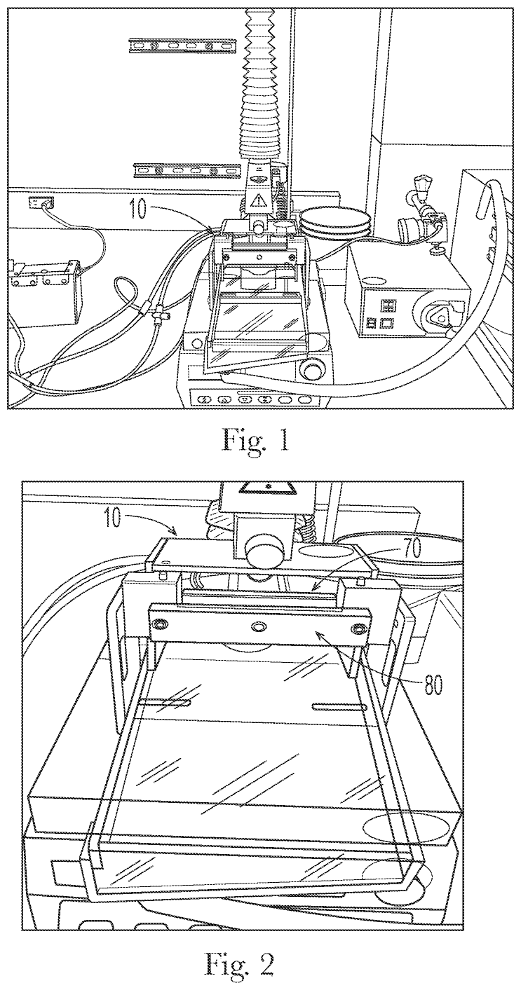

Install compression gig attachment 70 to the base plate 80 of the TA 10, as shown in FIG. 2.
Calibration: TA XT plus Texture Analyzer uses a 5 kg or 10 kg load cell requiring calibration. Standard calibration procedure is used.
Method sequence uses a speed setting of 16.7 mm/s and a distance setting of 167 mm.

### Test Method Execution

The compression gig attachment is connected to an air source and the pressure should be 50 psi. The compression gig is tested to ensure it opens and closes

### Sample Prep

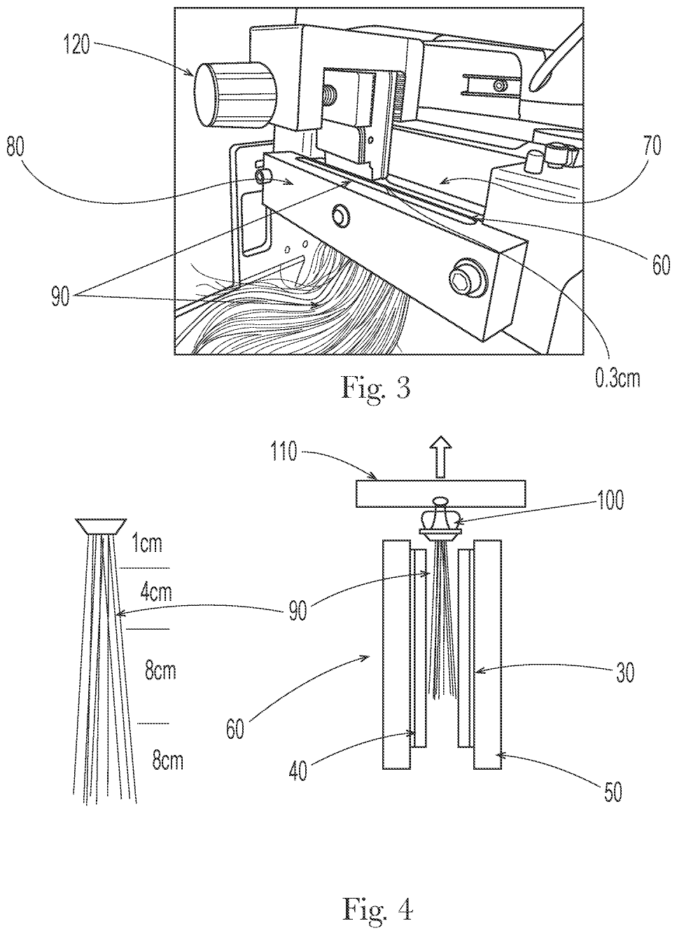

Artificial hair Tress Sizes: 4 g/200 mm ponytail is the standard tress sizes flattened using tress clip. Other tress sizes may be acceptable.
Dyed artificial sebum/soil in a vial placed in a water bath set at 43 C till turns liquid.
Add 40 mg of technical soil at the top region of the artificial tress, 1 cm from the epoxy root end with the aid of 200 ul pipette set at 45 ul.
For a cleaning substrate setup as shown in FIG. 4 attach a cleaning substrate (40) with one side of a double-sided tape (30) and the opposing side of the double sided tape (30) to a rectangular bar (50) to form a bar setup (60). As shown in FIG. 3 the bar setup (60) is attached to a compression gig attachment (70) connected to the base plate (80) of the TA.
As shown in FIG. 4, attach sample artificial hair tresses (90) to a clip (100) that helps to change hair orientation to a flat shape, and clamp clip (100) to the hair tress holder clamp (110), illustrated in FIG. 3 by the use of a black knob (120) to tighten the hair tress in position. Position hair tress (90) in between rectangular bar setup (50) and clamp the bars with the tress in between and the cleaning substrate attached to the bars together in a firm position with the aid of air pressure switch to hold the hair tress (90) firmly as shown in FIG. 3.
The distance from the base of the Bar represented as arrow (50) to the base of the white clip represented as arrow (100) should be 0.3 cm. (illustrated distance shown in FIG. 3)

### Artificial Soil Extraction and Analysis Method

### UV-VIS Extraction Procedure

Each cleaned hair tress is placed on top of 9 mm diameter clear rectangular impact resistance polycarbonate sheet with a calibrated Inkjet A4 Printer sheet paper placed at the base of the sheet to measure the three different cutting distances on each tresses.
The cleaned soiled hair tress is cut with the aid of a razor blade at a distance of 0.1 cm way from the epoxy at a length of 5 cm from the Root, 8 cm middle and 8 cm tip part of the hair.
Each part of the hair tress (List Parts) is inserted into a 50 ml centrifuge tube with 6 g of 2-Isopropanol (IPA) content.
The centrifuge tube 50 ml is inserted into a tissue culture rotator, and rotated for 10 minutes to extract the dyed soil or synthetic soil (Rotator is set at 6 rpm Speed).
Decant the extracted solution into a 5 ml 32 mm syringe attached to a Acrodisc Pall Corporation 25 mm w/ 0.2 um supor syringe filter.
Filter solution into cuvette and ready for absorbance measurement
Set the wavelength of UV-Vis to 518 nm.
Photometric value display is set to Absorbance (and not % T).
Blank the system with Isopropyl alcohol (IPA) A solution inserted in the sample holder.
Place decanted solution in cuvette in the sample holder.
Measure the absorbance of the solution, with the wavelength of the instrument (UV Spectrophotometer) set at 518 nm.
   Artificial Sebum Recovery is calculated from the calibration curve equation

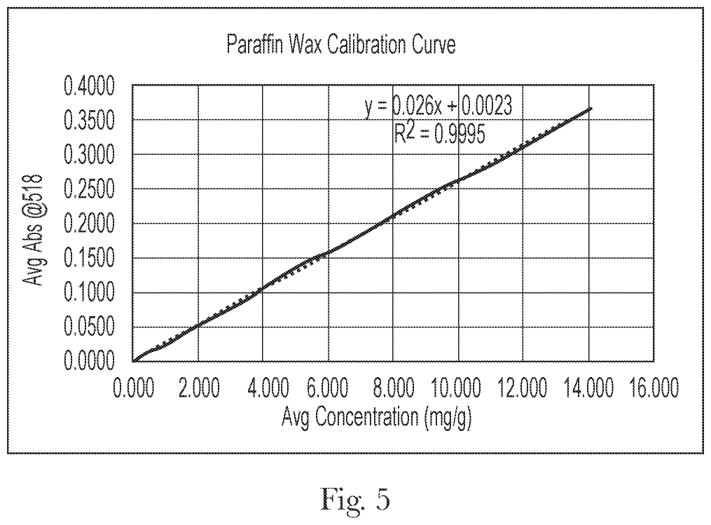

y=mx+c
X=Conc of solution (mg/g)
artificial sebum recovered (mg)=X*artificial sebum Applied
The Calibration Curve for soil is shown in Table 1 below and in FIG. 5.

| | | Avg | | | |
|---|---|---|---|---|---|
| | Avg | IPA | | | |
| | Artificial | Volume | Avg IPA | Avg | Avg |
| Sample | sebum | tested | mass | Concentration | Absorbance |
| # | [mg] | [ml] | (grams) | (mg/g) | @ 518 |
| 1 | 0 | 8 | 6.051 | 0.000 | 0.0000 |
| 2 | 2 | 8 | 6.011 | 0.333 | 0.0126 |
| 3 | 5 | 8 | 6.022 | 0.830 | 0.0218 |
| 4 | 9 | 8 | 6.025 | 1.494 | 0.0400 |
| 5 | 13.5 | 8 | 6.026 | 2.241 | 0.0597 |
| 6 | 19 | 8 | 6.026 | 3.154 | 0.0840 |
| 7 | 22 | 8 | 6.026 | 3.651 | 0.0967 |
| 8 | 30.5 | 8 | 6.029 | 5.059 | 0.1404 |
| 9 | 41 | 8 | 6.014 | 6.818 | 0.1792 |
| 10 | 49 | 8 | 6.017 | 8.144 | 0.2174 |
| 11 | 58.5 | 8 | 6.014 | 9.728 | 0.2578 |
| 12 | 65 | 8 | 6.020 | 10.797 | 0.2813 |
| 13 | 84.5 | 8 | 6.012 | 14.054 | 0.3653 |

### UV-Vis Setting/Calibration

### Turn on and Self-Check

Power on instrument to start Self-Check, which includes the following steps:
Turn on lamps→RAM Check→Start RTOS kernel→Initialize Comm. Port→Initialize Printer→Initialize AD→System position→Warm up.
Following Self-Check calibrate the instrument.

### Check the HF-0.5 Photometric Accuracy Standard

Set the wavelength to 465 nm. Ensure the cover is closed.
Make sure the photometric value display is set to Absorbance (and not % T).
Blank the system with nothing inserted in the sample holder.

Place HF-0.5 standard in the sample holder. Measure the absorbance value at 465 nm. The expected value at 465 nm is 0.4862±0.0073 Absorbance Units.

### Surface Tension Test

### Procedure for Measuring Surface Tension Using Kruss 100 Tensiometer

Surface tension is measured using a Kruss Model 100 Tensiometer (Kruss GMBH, Germany) or equivalent and Advance software. A Wilhelmy platinum probe PL01 was used with a wetting length of 40.2 mm. Both surface tension (mN/mi) and temperature (° C.) are recorded.

A cleaning composition is tested (volume) in 50 mL beaker. Samples are equilibrated to room temperature (21-24° C.) and then tested in duplicate. Water controls (±1 mN/m of expected value) are run before and after each composition to ensure the platinum probe is thoroughly clean.

Expected Value (water)=72.86 mN/m−(20° C.−Temp.)(−0.1514 mN/m/° C.)

The compositions of the present invention may include little or no surfactant; and surface tension should be less than 45 mN/m.

### Personal Care Compositions

The personal care compositions (or compositions) of the present invention comprise a cationic polymer and one or more of the components listed below.

The personal care compositions may be in the form of solutions, dispersion, spongers, foams, and other delivery mechanisms; and may fall into many consumer product categories, as described above.

### Cationic Polymers

The personal care composition comprises a cationic polymer. These cationic polymers may be naturally derived or naturally derived and then modified. Examples include polysaccharides such as cationic guar, cationic chitosan, cationic dextran, cationic cellulose, cationic cyclodextrin, cationic starch, cationic pectin, cationic polyglucan, and their derivatives. They also include cationic peptides and proteins. The cationic polymers may have at least one positively charged and/or pH dependent chargeable moiety that is at least one of a quaternary ammonium group, a primary amino group, a secondary amino group, or a tertiary amino group. A pH dependent chargeable primary amino group, secondary amino group, or tertiary amino group becomes predominantly positively charged when dissolved in an aqueous medium having a pH less than the pKa of the protonated primary amino group, secondary amino group, or tertiary amino group according to the Henderson-Hasselbach equation:

pH=pKa+log₁₀([A]/[HA⁺] where:
pKa=−log₁₀ Ka,
Ka is the dissociation constant of the reaction HA⁺+H₂O→A+H₃O⁺,
[A]=the concentration of a primary, secondary, or tertiary amine, and
[HA⁺]=the concentration of a protonated primary, secondary, or tertiary amine.

These cationic polymers can include at least one of (a) a cationic guar polymer, (b) a cationic non-guar galactomannan polymer, (c) a synthetic, non-crosslinked, cationic polymer, (d) a cationic cellulose polymer. Additionally, the cationic polymer can be a mixture of cationic polymers.

A synthetic cationic polymer may include several monomeric units, so they may be referred to as a copolymer rather than a homopolymer, which consists of a single type of monomeric unit. An example of a cationic homopolymer includes polyethylenimine. The polymers of the present disclosure may be a random copolymer. In one example, a polymer of the present disclosure may be water-soluble and/or water-dispersible, which means that the polymer does not, over at least a certain pH and concentration range, form a two-phase composition in water at 23° C.±2.2° C. In some embodiments, a polymer of the present invention comprises monomeric units such as those listed below:

a. Nonionic Monomeric Units

The nonionic monomeric units may be at least one of: nonionic hydrophilic monomeric units, nonionic hydrophobic monomeric units, or mixtures thereof.

Non-limiting examples of nonionic hydrophilic monomeric units suitable for the present invention include nonionic hydrophilic monomeric units derived from nonionic hydrophilic monomers at least one of: hydroxyalkyl esters of α,β-ethylenically unsaturated acids, such as hydroxyethyl or hydroxypropyl acrylates and methacrylates, glyceryl monomethacrylate, α,β-ethylenically unsaturated amides such as acrylamide, N,N-dimethylacrylamide, N,N-dimethylmethacrylamide, N-methylolacrylamide, α,β-ethylenically unsaturated monomers bearing a water-soluble polyoxyalkylene segment of the poly(ethylene oxide) type, such as poly(ethylene oxide) α-methacrylates (Bisomer S20W, S10W, etc., from Laporte) or α,ω-dimethacrylates, Sipomer BEM from Rhodia (ω-behenyl polyoxyethylene methacrylate), Sipomer SEM-25 from Rhodia (ω-tristyrylphenyl polyoxyethylene methacrylate), α,β-ethylenically unsaturated monomers which are precursors of hydrophilic units or segments, such as vinyl acetate, which, once polymerized, can be hydrolyzed in order to give rise to vinyl alcohol units or polyvinyl alcohol segments, vinylpyrrolidones, α,β-ethylenically unsaturated monomers of the ureido type, and in particular 2-imidazolidinone-ethyl methacrylamide (Sipomer WAM II from Rhodia), or mixtures thereof. In one example, the nonionic hydrophilic monomeric unit is derived from acrylamide.

Non-limiting examples of nonionic hydrophobic monomeric units suitable for the present invention include nonionic hydrophobic monomeric units derived from nonionic hydrophobic monomers that are at least one of: vinylaromatic monomers such as styrene, alpha-methylstyrene, vinyltoluene, vinyl halides or vinylidene halides, such as vinyl chloride, vinylidene chloride, C₁-C₁₂ alkylesters of α,β-monoethylenically unsaturated acids such as methyl, ethyl or butyl acrylates and methacrylates, 2-ethylhexyl acrylate, vinyl esters or allyl esters of saturated carboxylic acids, such as vinyl or allyl acetates, propionates, versatates, stearates, α,β-monoethylenically unsaturated nitriles containing from 3 to 12 carbon atoms, such as acrylonitrile, methacrylonitrile, α-olefins such as ethylene, conjugated dienes, such as butadiene, isoprene, chloroprene, or mixtures thereof.

b. Cationic Monomeric Units

Non-limiting examples of cationic or pH dependent chargeable monomeric units suitable for the present invention include amine containing monomeric units derived from monomers that are at least one of: N,N-(dialkylamino-ω-alkyl)amides of α,β-monoethylenically unsaturated carboxylic acids, such as N,N-dimethylaminomethyl-acrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride (METAC) or methyl sulfate, and acryloyloxyethyltrimethylammonium chloride (AETAC); 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N1-(3-(2((3-methacrylamidopropyl)dimethylammino)-acetamido)propyl)-N1, N1, N3, N3, N3-pentamethylpropane-1,3-diaminium chloride (TRIQUAT or TQ), or mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from TQ.

In embodiments, the non-ionic monomers are selected from acrylamide derivatives from the group consisting of: acrylamide, mono-alkyl substituted acrylamide, symmetrical or asymmetrical, di-N-alkyl substituted acrylamide derivatives, methacrylamide, mono-alkyl substituted methacrylamide, symmetrical or asymmetrical, di-N-alkyl substituted methacrylamide derivatives and mixtures thereof.

In another example, the acrylamide derivatives of the present invention are at least one of: N,N-dimethylacrylamide (NDMAAM), acrylamide, methyl acrylamide, ethylacrylamide, N,N-diethylacrylamide, methacrylamide, N,N-dimethyl methacrylamide, or mixtures thereof.

Further examples of cationic monomeric units suitable for the present invention include cationic or pH dependent chargeable monomeric units that are at least one of: N,N-(dialkylamino-ω-alkyl)amides of α,β-monoethylenically unsaturated carboxylic acids, such as N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammonium ethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride or methyl sulfate, and acryloyloxyethyltrimethylammonium chloride; 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N¹-(3-(2((3-methacrylamidopropyl)dimethylammino)-acetamido)propyl)-N¹, N¹, N³, N³, N³-pentamethylpropane-1,3-diaminium chloride (TRIQUAT or TQ), or mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from TQ.

In embodiments, the pH dependent chargeable monomeric units are at least one of: dimethylaminoethyl (meth)acrylate, dimethylaminopropyl (meth)acrylate, di-tert-butylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine and vinyl imidazole, and mixtures thereof.

In embodiments, the cationic monomeric units are at least one of: trimethylammonium ethyl (meth)acrylate bromide, chloride or methyl sulfate, trimethylammonium ethyl (meth)acrylate bromide, chloride or methyl sulfate, trimethylammonium ethyl (meth)acrylate bromide, chloride or methyl sulfate, dimethylaminoethyl (meth)acrylate benzyl chloride, 4-benzoylbenzyl dimethylammoniumethyl (meth)acrylate bromide, chloride or methyl sulfate, trimethylammonium ethyl (meth)acrylamido bromide, chloride, or methyl sulfate, trimethylammonium propyl (meth)acrylamido bromide, chloride, or methyl sulfate, vinyl benzyl trimethyl ammonium bromide, chloride or methyl sulfate, diallyldimethyl ammonium chloride, 1-ethyl-2-vinylpyridinium bromide, chloride or methyl sulfate, 4-vinylpyridinium bromide, chloride or methyl sulfate, and mixtures thereof.

The personal care composition may comprise a cationic guar polymer, which is a cationically substituted galactomannan (guar) gum derivatives. Guar gum for use in preparing these guar gum derivatives is typically obtained as a naturally occurring material from the seeds of the guar plant. The guar molecule itself is a straight chain mannan, which is branched at regular intervals with single membered galactose units on alternative mannose units. The mannose units are linked to each other by means of β(1-4) glycosidic linkages. The galactose branching arises by way of an α(1-6) linkage. Cationic derivatives of the guar gums are obtained by reaction between the hydroxyl groups of the polygalactomannan and reactive quaternary ammonium compounds. The degree of substitution of the cationic groups onto the guar structure should be sufficient to provide the requisite cationic charge density described above.

The cationic polymer, may include but is not limited to a cationic guar polymer; wherein a guar polymer may have a weight average molecular weight of less than about 10 million g/mol, or from about 400 thousand to about 10 million g/mol, or from about 500 thousand to about 5 million g/mol, or from about 750 thousand to about 3 million g/mol, or from about 1 million to about 2 million g/mol. The cationic guar polymer may have a charge density of from about 0.4 to about 4.0 meq/g, or from about 0.6 to about 3.0 meq/g, or from about 0.75 to about 2.5 meq/g; or from about 1.0 meq/g to about 2.0 meq/g.

Suitable cationic guar polymers include cationic guar gum derivatives, such as guar hydroxypropyltrimonium chloride. The cationic guar polymer may be a guar hydroxypropyltrimonium chloride. Specific examples of guar hydroxypropyltrimonium chlorides include the Jaguar® series commercially available from Solvay (Solvay USA Inc., Cincinnati, Ohio), for example Jaguar® C-500, commercially available from Solvay. Jaguar® C-500 has a charge density of 0.8 meq/g and a molecular weight of 500,000 g/mol. Other suitable guar hydroxypropyltrimonium chloride are: guar hydroxypropyltrimonium chloride which has a charge density of about 1.3 meq/g and a molecular weight of about 500,000 g/mol and is available from Solvay as Jaguar® Optima. Other suitable guar hydroxypropyltrimonium chloride are: guar hydroxypropyltrimonium chloride which has a charge density of about 0.7 meq/g and a molecular weight of about 1,500,000 g/mol and is available from Solvay as Jaguar® Excel. Other suitable guar hydroxypropyltrimonium chloride are: guar hydroxypropyltrimonium chloride which has a charge density of about 1.1 meq/g and a molecular weight of about 500,000 g/mol and is available from ASI.

Other suitable guar hydroxypropyltrimonium chloride are: Hi-Care 1000, which has a charge density of about 0.7 meq/g and a molecular weight of about 600,000 g/mole and is available from Solvay; N-Hance 3269 and N-Hance 3270, which have a charge density of about 0.7 meq/g and a molecular weight of about 425,000 g/mol and are available from ASI; N-Hance 3196, which has a charge density of about 0.8 meq/g and a molecular weight of about 1,100,000 g/mol and is available from ASI. BF-13, which is a borate (boron) free guar of charge density of about 1.1 meq/g and molecular weight of about 800,000 and BF-17, which is a borate (boron) free guar of charge density of about 1.7 5 meq/g and molecular weight of about 800,000 both available from ASI. Another suitable guar hydroxypropyltrimonium chloride is Dehyquart Guar HP available from BASF.

The personal care compositions of the present invention may comprise a galactomannan polymer derivative having a mannose to galactose ratio of greater than 2:1 on a monomer to monomer basis, the galactomannan polymer derivative at least one of a cationic galactomannan polymer derivative and an amphoteric galactomannan polymer derivative having a net positive charge. As used herein, the term “cationic galactomannan” refers to a galactomannan polymer to which a cationic group is added. The term “amphoteric galactomannan” refers to a galactomannan polymer to which a cationic group and an anionic group are added such that the polymer has a net positive charge.

Galactomannan polymers are present in the endosperm of seeds of the Leguminosae family. Galactomannan polymers are made up of a combination of mannose monomers and galactose monomers. The galactomannan molecule is a straight chain mannan branched at regular intervals with single membered galactose units on specific mannose units. The mannose units are linked to each other by means of β (1-4) glycosidic linkages. The galactose branching arises by way of an α (1-6) linkage. The ratio of mannose monomers to galactose monomers varies according to the species of the plant and also is affected by climate. Non-Guar Galactomannan polymer derivatives of the present invention have a ratio of mannose to galactose of greater than 2:1 on a monomer to monomer basis. Suitable ratios of mannose to galactose can be greater than about 3:1, and the ratio of mannose to galactose can be greater than about 4:1. Analysis of mannose to galactose ratios is well known in the art and is typically based on the measurement of the galactose content.

The gum for use in preparing the non-guar galactomannan polymer derivatives is typically obtained as naturally occurring material such as seeds or beans from plants. Examples of various non-guar galactomannan polymers include but are not limited to Tara gum (3 parts mannose/1 part galactose), Locust bean or Carob (4 parts mannose/1 part galactose), and Cassia gum (5 parts mannose/1 part galactose).

The non-guar galactomannan polymer derivatives may have a molecular weight from about 400,000 g/mol to about 10,000,000 g/mol, and/or from about 500,000 g/mol to about 5,000,000 g/mol.

The personal care compositions of the invention can also include galactomannan polymer derivatives which have a cationic charge density from about 0.1 meq/g to about 3.0 meq/g. The galactomannan polymer derivatives may have a cationic charge density from about 0.6 meq/g to about 3 meq/g. The degree of substitution of the cationic groups onto the galactomannan structure should be sufficient to provide the requisite cationic charge density.

The galactomannan polymer derivative can be a cationic derivative of the non-guar galactomannan polymer, which is obtained by reaction between the hydroxyl groups of the polygalactomannan polymer and reactive quaternary ammonium compounds.

Alternatively, the galactomannan polymer derivative can be an amphoteric galactomannan polymer derivative having a net positive charge, obtained when the cationic galactomannan polymer derivative further comprises an anionic group.

The cationic non-guar galactomannan can have a ratio of mannose to galactose greater than about 4:1, a molecular weight of about 400,000 g/mol to about 10,000,000 g/mol, and/or from about 500,000 g/mol to about 10,000,000 g/mol, and/or from about 750,000 g/mol to about 3,000,000 g/mol, and/or from about 1,000,000 g/mol to about 2,000,000 g/mol and a cationic charge density from about 0.4 meq/g to about 4 meq/g, and/or from 0.6 meq/g to about 3 meq/g and can be derived from a cassia plant.

The personal care compositions of the present invention can also include ‘SoftCAT’ or ‘UCare’ polymers: The SoftCAT™ SL (SoftCAT™ SL, INCI name: Polyquaternium-67; Ballarin et al., 2011) constitute a family of high viscosity quaternized hydroxyethyl cellulose (HEC) polymers, with low cationic substitution, of trimethyl ammonium and dimethyldodecyl ammonium. “UCARE™ polymers (INCI Name: Polyquaternium-10) are polymeric, quaternary ammonium salts of hydroxyethylcellulose reacted with trimethyl ammonium substituted epoxide. The cellulosic backbone is derived from natural, renewable resources.

The synthetic cationic polymers of the present invention can be made by a wide variety of techniques, including bulk, solution, emulsion, or suspension polymerization. Polymerization methods and techniques for polymerization are described generally in Encyclopedia of Polymer Science and Technology, Interscience Publishers (New York), Vol. 7, pp. 361-431 (1967), and Kirk-Othmer Encyclopedia of Chemical Technology, 3rd edition, Vol 18, pp. 740-744, John Wiley & Sons (New York), 1982, both incorporated by reference herein. See also Sorenson, W. P. and Campbell, T. W., Preparative Methods of Polymer Chemistry. 2nd edition, Interscience Publishers (New York), 1968, pp. 248-251, incorporated by reference herein, for general reaction techniques suitable for the present invention. In one example, the polymers are made by free radical copolymerization, using water soluble initiators. Suitable free radical initiators include, but are not limited to, thermal initiators, redox couples, and photochemical initiators. Redox and photochemical initiators may be used for polymerization processes initiated at temperatures below about 30° C. (86° F.). Such initiators are described generally in Kirk-Othmer Encyclopedia of Chemical Technology, 3rd edition, John Wiley & Sons (New York), Vol. 13, pp. 355-373 (1981), incorporated by reference herein. Typical water soluble initiators that can provide radicals at 30° C. or below include redox couples, such as potassium persulfate/silver nitrate, and ascorbic acid/hydrogen peroxide. In one example, the method utilizes thermal initiators in polymerization processes conducted above 40° C. (104° F.). Water soluble initiators that can provide radicals at 40° C. (104° F.) or higher can be used. These include, but are not limited to, hydrogen peroxide, ammonium persulfate, and 2,2′-azobis(2-amidinopropane) dihydrochloride. In one example, water soluble starting monomers are polymerized in an aqueous alcohol solvent at 60° C. (140° F.) using 2,2′-azobis(2-amidinopropane) dihydrochloride as the initiator.

### Liquid Personal Care Compositions

Liquid personal care compositions may include an aqueous carrier, which can be present at a level of from about 80% or greater. The aqueous carrier may comprise water, or a miscible mixture of water and organic solvent. Non-aqueous carrier materials may also be employed.

The personal care composition may be applied by a variety of means, including by rubbing, wiping or dabbing with hands or fingers, or by means of an implement and/or delivery enhancement device. Non-limiting examples of implements include a sponge or sponge-tipped applicator, a mesh shower puff, a swab, a brush, a wipe (e.g., wash cloth), a loofah, and combinations thereof. Non-limiting examples of delivery enhancement devices include mechanical, electrical, ultrasonic and/or other energy devices, including a brush-like device that adapts to tangles in hair and allows for easy removal of hair post-groom using uniquely shaped, anisotropic papillae as described in US20190142151A1. The personal care composition may be sold together with such an implement or device. Alternatively, an implement or device can be sold separately but contain indicium to indicate usage with a personal care composition. Implements and delivery devices can employ replaceable portions (e.g., the skin interaction portions), which can be sold separately or sold together with the personal care composition in a kit.

### Optional Ingredients

In the present invention, a personal care composition may further comprise one or more optional ingredients, including benefit agents. Suitable benefit agents include, but are not limited to conditioning agents, anti-dandruff agents, chelating agents, and natural oils such as sunflower oil or castor oil. Additional suitable optional ingredients include but are not limited to perfumes, perfume microcapsules, colorants, particles, anti-microbials, foam busters, anti-static agents, rheology modifiers and thickeners, suspension materials and structurants, pH adjusting agents and buffers, preservatives, pearlescent agents, sensates, anti-dandruff agents, propellants, solvents, diluents, anti-oxidants, vitamins and combinations thereof. In the present invention, the composition may have from about 0.5% to about 2% of a perfume.

Such optional ingredients should be physically and chemically compatible with the components of the composition, and should not otherwise unduly impair product stability, aesthetics, or performance. The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

Personal care compositions can also include one or more humectants. Examples of such humectants can include polyhydric alcohols. Further, humectants such as glycerin can be included the personal care composition as a result of production or as an additional ingredient. Including additional humectant can result in a number of benefits such as improvement in hardness of the personal care composition, decreased water activity of the personal care composition, and reduction of a weight loss rate of the personal care composition over time due to water evaporation.

### Chelating Agents

Personal care compositions of the present invention can also comprise a chelant. Suitable chelants include those listed in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference. When related to chelants, the term “salts and derivatives thereof” means the salts and derivatives comprising the same functional structure (e.g., same chemical backbone) as the chelant they are referring to and that have similar or better chelating properties.

Chelating agents can be incorporated in the compositions herein in amounts ranging from 0.001% to 10.0% by weight of the total composition, preferably 0.01% to 2.0%.

Nonlimiting chelating agent classes include carboxylic acids, aminocarboxylic acids, including aminoacids, phosphoric acids, phosphonic acids, polyphosphonic acids, polyethyleneimines, polyfunctionally-substituted aromatic, their derivatives and salts.

Nonlimiting chelating agents include the following materials and their salts. Ethylenediaminetetraacetic acid (EDTA), ethylenediaminetriacetic acid, ethylenediamine-N,N′-disuccinic acid (EDDS), ethylenediamine-N,N′-diglutaric acid (EDDG), salicylic acid, aspartic acid, glutamic acid, glycine, malonic acid, histidine, diethylenetriaminepentaacetate (DTPA), N-hydroxyethylethylenediaminetriacetate, nitrilotriacetate, ethylenediaminetetrapropionate, triethylenetetraaminehexaacetate, ethanoldiglycine, propylenediaminetetracetic acid (PDTA), methylglycinediacetic acid (MODA), diethylenetriaminepentaacetic acid, methylglycinediacetic acid (MGDA), N-acyl-N,N′,N′-ethylenediaminetriacetic acid, nitrilotriacetic acid, ethylenediaminediglutaric acid (EDGA), 2-hydroxypropylenediamine disuccinic acid (HPDS), glycinamide-N, N′-disuccinic acid (GADS), 2-hydroxypropylenediamine-N—N′-disuccinic acid (HPDDS), N-2-hydroxyethyl-N,N-diacetic acid, glyceryliminodiacetic acid, iminodiacetic acid-N-2-hydroxypropyl sulfonic acid, aspartic acid N-carboxymethyl-N-2-hydroxypropyl-3-sulfonic acid, alanine-N,N′-diacetic acid, aspartic acid-N,N′-diacetic acid, aspartic acid N-monoacetic acid, iminodisuccinic acid, diamine-N,N′-dipolyacid, monoamide-N,N′-dipolyacid, diaminoalkyldi(sulfosuccinic acids) (DDS), ethylenediamine-N—N′-bis (ortho-hydroxyphenyl acetic acid)), N,N′-bis(2-hydroxybenzyl)ethylenediamine-N, N′-diacetic acid, ethylenediaminetetraproprionate, triethylenetetraaminehexacetate, diethylenetriaminepentaacetate, dipicolinic acid, ethylenedicysteic acid (EDC), ethylenediamine-N,N′-bis(2-hydroxyphenylacetic acid) (EDDHA), glutamic acid diacetic acid (GLDA), hexadentateaminocarboxylate (HBED), polyethyleneimine, 1-hydroxydiphosphonate, aminotri(methylenephosphonic acid) (ATMP), nitrilotrimethylenephosphonate (NTP), ethylenediaminetetramethylenephosphonate, diethylenetriaminepentamethylenephosphonate (DTPMP), ethane-1-hydroxydiphosphonate (HEDP), 2-phosphonobutane-1,2,4-tricarboxylic acid, polyphosphoric acid, sodium tripolyphosphate, tetrasodium diphosphate, hexametaphosphoric acid, sodium metaphosphate, phosphonic acid and derivatives, Aminoalkylen-poly(alkylenphosphonic acid), aminotri(1-ethylphosphonic acid), ethylenediaminetetra(1-ethylphosphonic acid), aminotri(1-propylphosphonic acid), aminotri(isopropylphosphonic acid), ethylenediaminetetra(methylenephosphonic acid) (EDTMP), 1,2-dihydroxy-3,5-disulfobenzene.

The carrier useful the personal care compositions of the present invention may include water and water solutions of lower alkyl alcohols and polyhydric alcohols. The lower alkyl alcohols useful herein are monohydric alcohols having 1 to 6 carbons, in one aspect, ethanol and isopropanol. Exemplary polyhydric alcohols useful herein include propylene glycol, hexylene glycol, glycerin, and propane diol.

### Propellant or Blowing Agent

The personal care composition described herein may comprise from about from about 1% to about 10% propellant or blowing agent, alternatively from about 2% to about 8% propellant, by weight of the personal care composition.

The propellant or blowing agent may comprise one or more volatile materials, which in a gaseous state, may carry the other components of the personal care composition in particulate or droplet form or as a foam. The propellant or blowing agent may have a boiling point within the range of from about −45° C. to about 5° C. The propellant or blowing agent may be liquefied when packaged in convention aerosol containers under pressure. The rapid boiling of the propellant or blowing agent upon leaving the aerosol foam dispenser may aid in the atomization or foaming of the other components of the personal care composition.

Aerosol propellants or blowing agents which may be employed in an aerosol composition of the present invention may include the chemically-inert hydrocarbons such as propane, n-butane, isobutane, cyclopropane, and mixtures thereof, as well as halogenated hydrocarbons such as dichlorodifluoromethane, 1,1-dichloro-1,1,2,2-tetrafluoroethane, 1-chloro-1,1-difluoro-2,2-trifluoroethane, 1-chloro-1,1-difluoroethane, 1,1-difluoroethane, dimethyl ether, monochlorodifluoromethane, trans-1,3,3,3-tetrafluoropropene, and mixtures thereof. The propellant or blowing agent may comprise hydrocarbons such as isobutane, propane, and butane—these materials may be used for their low ozone reactivity and may be used as individual components where their vapor pressures at 21.1° C. range from about 1.17 Bar to about 7.45 Bar, alternatively from about 1.17 Bar to about 4.83 Bar, and alternatively from about 2.14 Bar to about 3.79 Bar.

### Applicator

In the present invention, personal care composition may be dispensed from an applicator for dispensing directly to the scalp area. Dispensing directly onto the scalp via a targeted delivery applicator enables deposition of the non-diluted cleaning agents directly where the cleaning needs are highest. This also minimizes the risk of eye contact with the cleansing solution.

The applicator is attached or can be attached to a bottle containing the cleansing personal care composition. The applicator can consist of a base that holds or extends to a single or plurality of tines. The tines have openings that may be at the tip, the base or at any point between the tip and the base. These openings allow for the product to be distributed from the bottle directly onto the hair and/or scalp.

Alternatively, the applicator can also consist of brush-like bristles attached or extending from a base. In this case product would dispense from the base and the bristles would allow for product distribution via the combing or brushing motion. In embodiments, the applicator could take the form a brush-like device that adapts to tangles in hair and allows for easy removal of hair post-groom using uniquely shaped bristles that mimic anisotropic papillae like those found a feline tongue, as described in US20190142151A1.

Applicator and tine design and materials can also be optimized to enable scalp massage. In this case it would be beneficial for the tine or bristle geometry at the tips to be more rounded similar to the roller ball applicator used for eye creams. It may also be beneficial for materials to be smoother and softer; for example, metal or metal-like filaments.

### Cleaning Composition Examples

Inventive and comparative cleaning composition examples were made according to the following process.

High quality purified and deionized water was added to a 125 ml High Density Polyethylene (HDPE) wide-mouth bottle. The specified amount of polymer (dry or solution) was added to the water with sufficient mixing to create a mild vortex. This was achieved with a Polytetrafluoroethylene-coated (PTFE), magnetic stir bar and Magnetic Stirrer (example VWR (25.4×25.4 cm) Stirrer, Ceramic, 120V 10.4 amps 50 watts, Catalogue #97042-746). The polymer was dispersed in water for 10 minutes before the solvents were added at the specified levels. The solutions were mixed for 15 minutes before using for testing and were used within 30 days for the performance testing.

TABLE
Personal Care Composition Examples containing cleaning polymer¹

| | I | II | III | IV | V |
|---|---|---|---|---|---|
| Ingredients | (Inventive) | (Comparative) | Inventive | (Comparative) | (Comparative) |
| Vinylpyrrolidone/Dimethylamino- | 0.25 | | 0.25 | | |
| ethylmethacrylate Copolymer | | | | | |
| Hydrophobically modified | | | | | |
| cationic cellulose SoftCAT ™ | | | | | |
| Ethoxy hydroxypropyl cationic | | | | | |
| cellulose UCARE ™ | | | | | |
| Mirapol surf s-210 | | | | | 0.1 |
| 2-(2-Butoxyethoxy)ethanol | 0.83 | | 0.83 | | |
| PPG-3 Methyl Ether | 0.83 | | 0.83 | | |
| Propylene Glycol Propyl Ether | 0.83 | | 0.83 | | |
| Propylene Glycol | | | | | 0.25 |
| Ethyl Alcohol, 40B | 10.0 | | 10.0 | | |
| Hexyl cellosolve | | | | | 0.6 |
| Isopropyl alcohol | 1.00 | | 1.00 | | 1.0 |
| VIDET EGM polymer surfactant | | | | | 0.5 |
| Citric Acid | 0.05 | | 0.05 | | |
| Sodium Citrate, Dihydrate | 0.05 | | 0.05 | | |
| Ammonium Hydroxide (30%) | | | | | 0.3 (0.09) |
| Polyoxyethylene (20) Sorbitan | 0.09 | | 0.09 | | |
| Monopalmitate | | | | | |
| POLYSORBATE (80) (POE (20) | 0.03 | | 0.03 | | |
| Sorbitan Monooleate | | | | | |
| Secondary Alkane Sulfonate, | | | | | 0.4 (0.12) |
| sodium salt (30%) | | | | | |
| Hostapur SAS 30 Clariant | | | | | |
| Perfume | 0.10 | | 0.10 | | 0.05 |
| Water | QS | 100 | QS | 100 | QS |
| Receiver Substrate | Cotton | Cotton | Skin | Skin | Cotton |
| | | | Surrogate | Surrogate | |
| wt/wt % | I | II | III | IV | V |
| Test A (Artificial Sebum/soil | 29 (±2.71) | 14 (±1.14) | 17 (±1.29) | 8 (±2.36) | 20 (±3.24) |
| Removal-Transfer) % Removed | | | | | |
| Test B (Artificial Sebum/soil | 54 (±0.02) | 60 (±0.02) | 62 (±0.02) | 63 (±0.02) | 61 (±0.01) |
| Spread-Zone: Root) % Spread | | | | | |
| Test B (Artificial Sebum | 17 (±0.02) | 26 (±0.02) | 21 (±0.02) | 29 (±0.02) | 19 (±0.01) |
| Spread-Zone: Length) % Spread | | | | | |

| | VI | VII | VIII | IX |
|---|---|---|---|---|
| Ingredients | (Inventive) | (Inventive) | (Inventive) | (Comparative) |
| Vinylpyrrolidone/Dimethylamino- | | | 0.125 | |
| ethylmethacrylate Copolymer | | | | |
| Hydrophobically modified | | 0.25 | 0.125 | |
| cationic cellulose SoftCAT ™ | | | | |
| Ethoxy hydroxypropyl cationic | 0.25 | | | |
| cellulose UCARE ™ | | | | |
| Mirapol surf s-210 | | | | 0.1 |
| 2-(2-Butoxyethoxy)ethanol | 0.83 | 0.83 | 0.83 | |
| PPG-3 Methyl Ether | 0.83 | 0.83 | 0.83 | |
| Propylene Glycol Propyl Ether | 0.83 | 0.83 | 0.83 | |
| Propylene Glycol | | | | 0.25 |
| Ethyl Alcohol, 40B | 10.0 | 10.0 | 10.0 | |
| Hexyl cellosolve | | | | 0.6 |
| Isopropyl alcohol | 1.00 | 1.00 | 1.00 | 1.0 |
| VIDET EGM polymer surfactant | | | | 0.5 |
| Citric Acid | 0.05 | 0.05 | 0.05 | |
| Sodium Citrate, Dihydrate | 0.05 | 0.05 | 0.05 | |
| Ammonium Hydroxide (30%) | | | | 0.3 (0.09) |
| Polyoxyethylene (20) Sorbitan | 0.09 | 0.09 | 0.09 | |
| Monopalmitate | | | | |
| POLYSORBATE (80) (POE (20) | 0.03 | 0.03 | 0.03 | |
| Sorbitan Monooleate | | | | |
| Secondary Alkane Sulfonate, | | | | 0.4 (0.12) |
| sodium salt (30%) | | | | |
| Hostapur SAS 30 Clariant | | | | |
| Perfume | 0.10 | 0.10 | 0.10 | 0.05 |
| Water | QS | QS | QS | QS |
| Receiver Substrate | Cotton | Cotton | Cotton | Skin |
| | | | | Surrogate |
| wt/wt % | VI | VII | VIII | X |
| Test A (Artificial Sebum/soil | 39 (+4.69%) | 36 (+3.28%) | 34 (±2.71%) | 10 (±0.24) |
| Removal-Transfer) % Removed | | | | |
| Test B (Artificial Sebum/soil | 38 (±0.7) | 25 (±0.01%) | 41 (±0.02%) | 64 (±0.01) |
| Spread-Zone: Root) % Spread | | | | |
| Test B (Artificial Sebum | 23 (±0.047) | 26 (±0.02) | 25 (±0.04%) | 26 (±0.01) |
| Spread-Zone: Length) % Spread | | | | |

¹numerical values are wt/wt %.

The TABLE shows inventive and comparative examples of rinse free cleaning compositions. Comparative examples II and IV, comprised only water and were applied to Synthetic PVC Hair Tresses and tested with two different substrates (cotton and a skin surrogate) for cleaning (artificial sebum removal and spread) while comparative examples V and IX, which comprised a binary mixture of two nonalcoholic glycol ether solvents (i.e. 0.25% propylene glycol n-butyl ether and 0.65% Hexyl cellosolve) along with a polymer (Mirapol surf s-210), were also applied to Synthetic PVC Hair Tresses and tested with two different substrates (cotton and a skin surrogate) for cleaning. As shown in the TABLE, inventive examples I, VI, VII, VIII clean significantly better (i.e. 29, 39, 36, and 34% artificial sebum removal) than comparative examples II (water alone) and V (a binary mixture of two nonalcoholic glycol ether solvents along with a polymer)—i.e. 14 and 20% artificial sebum removal, when used with a cotton substrate. When the cleaning substrates were changed from cotton to a skin surrogate inventive example III removed artificial sebum significantly better (17% vs 8% than comparative example IV (water alone) and significantly better (17% vs. 10%) than comparative example IX (a binary mixture of two nonalcoholic glycol ether solvents along with a polymer).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) about 0.01% to about 1.0% polymer, by weight of said composition, wherein the polymer is cationic either through a quaternary amine or pH dependent chargeable mono-, di-, or tri-alkyl amine; wherein the cationic polymer has a ratio of cationic or pH dependent chargeable monomers to noncationic or non-chargeable monomers of from about 1:99 to about 20:80; wherein the polymer has a molecular weight of from about 10,000 to about 10,000,000;
b) about 1.0% to about 30%, by weight of said composition, of a triple solvent system with surface tension of less than about 45 mN/m; comprising a first solvent, a second solvent, and a third solvent, wherein each of said first solvent, said second solvent, and said third solvent are different; wherein HLB value of the solvents is from about 6.25 to about 8.5;
wherein the composition has a viscosity of about 1.0 cps to about 10 cps;
wherein the composition is substantially surfactant free;
wherein the composition removes at least about 25% artificial sebum measured by the Extraction and UV-VIS analysis methods, and spreads at least about 20%, as determined by root % spread plus length % spread, of the non-removed artificial sebum from the roots to the remaining hair length as measured by SSRT methods.

2. The personal care composition according to claim 1, wherein the polymer has a molecular weight of from about 200,000 to about 3,000,000.

3. The personal care composition according to claim 1, wherein the composition removes at least about 35% artificial sebum measured by the Extraction and UV-VIS analysis methods.

4. The personal care composition according to claim 1, wherein the composition removes at least about 45% artificial sebum measured by the Extraction and UV-VIS analysis methods.

5. The personal care composition according to claim 1, wherein the composition spreads at least about 35%, as determined by root % spread plus length % spread, of the non-removed artificial sebum from the roots to the remaining hair length as measured by SSRT methods.

6. The personal care composition according to claim 1, wherein the composition spreads at least about 50%, as determined by root % spread plus length % spread, of the non-removed artificial sebum from the roots to the remaining hair length as measured by SSRT methods.

7. The personal care composition according to claim 1, wherein the composition spreads at least about 75%, as determined by root % spread plus length % spread, of the non-removed artificial sebum from the roots to the remaining hair length as measured by SSRT methods.

8. The personal care composition according to claim 1, wherein the composition spreads at least about 85%, as determined by root % spread plus length % spread, of the non-removed artificial sebum from the roots to the remaining hair length as measured by SSRT methods.

9. The personal care composition according to claim 1, wherein the composition comprises about 1.0% to about 25%, by weight of said composition, of the triple solvent system.

10. The personal care composition according to claim 1, wherein the composition comprises about 1.0% to about 20%, by weight of said composition, of the triple solvent system.

11. The personal care composition according to claim 1, wherein the composition comprises about 10% to about 20%, by weight of said composition, of the triple solvent system.

12. The personal care composition according to claim 1, wherein the composition comprises about 0.02% to about 2% cationic polymer, by weight of said composition.

13. The personal care composition according to claim 1, wherein the first and second solvents are independently at least one of at least one of propylene glycol n-butyl ether (dowanol PnB), tripropylene glycol methyl ether (dowanol TPM), dipropylene glycol n-propyl ether (dowanol DPnP), dipropylene glycol n-butyl ether (dowanol DPnB), tripropylene glycol n-butyl ether (dowanol TPnB), diethylene glycol n-butyl ether (Butyl carbitol), diethylene glycol hexyl ether (hexyl Carbitol), diethylene glycol n-butyl ether acetate (butyl carbitol acetate), ethylene glycol hexyl ether (hexyl cellosolve), triethylene glycol methyl ether (methoxytriglycol), triethylene glycol ethyl ether (ethoxytriglycol), triethylene glycol n-butyl ether (butoxytriglycol), Ucar filmer ibt, trimethylnonanol, propylene glycol diacetate (dowanol PGDA), or dipropylene glycol methyl ether (dowanol DPM).

14. The personal care composition according to claim 13, wherein the third solvent is at least one of propylene glycol n-butyl ether (dowanol PnB), tripropylene glycol methyl ether (dowanol TPM), dipropylene glycol n-propyl ether (dowanol DPnP), dipropylene glycol n-butyl ether (dowanol DPnB), tripropylene glycol n-butyl ether (dowanol TPnB), diethylene glycol n-butyl ether (Butyl carbitol), diethylene glycol hexyl ether (hexyl Carbitol), diethylene glycol n-butyl ether acetate (butyl carbitol acetate), ethylene glycol hexyl ether (hexyl cellosolve), triethylene glycol methyl ether (methoxytriglycol), triethylene glycol ethyl ether (ethoxytriglycol), triethylene glycol n-butyl ether (butoxytriglycol), Ucar filmer ibt, trimethylnonanol, propylene glycol diacetate (dowanol PGDA), dipropylene glycol methyl ether (dowanol DPM), ethanol, isopropanol, isopropyl myristate, propylene glycol, dipropylene glycol, hexylene glycol, or ethoxy diglycol, and 1,2 hexane diol.

15. The personal care composition according to claim 1, wherein the polymer is at least one of:
(i) polymers having the following formula: 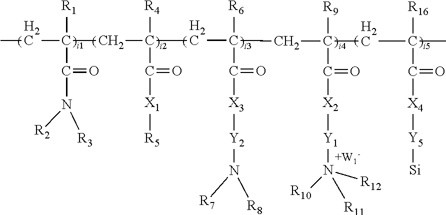
wherein
R₁, R₂, R₃, R₄, R₆, R₇, R₈, R₉, and R₁₆ are each independently selected from H or C₁-C₆ alkyl;
X₁, X₂, X₃ and X₄ are each independently selected from O or NH;
R₅ is C₈-C₃₀ n- or iso-alkyl;
Y₁, Y₂, and Y₅ are each independently selected from —(CH₂)ₘ—, wherein m is 1-30;
R₁₀, R₁₁, and R₁₂ are each independently selected from C₁-C₆ alkyl; preferably R₁₀, R₁₁, and R₁₂ are each independently selected from methyl, ethyl and propyl;
W₁⁻ is a counter ion; preferably W₁⁻ is selected from Cl⁻, Br⁻, I⁻, HSO₄⁻, CH₃SO₄⁻, C₂H₅SO₄⁻, or OH⁻;
Si is a silicone or derivative thereof, preferably Si is at least one of a polydimethylsiloxane, an aminosilicone, a cationic silicone, a silicone polyether, a cyclic silicone, a fluorinated silicone and mixtures thereof; preferably Si is polydimethylsiloxane; preferably Si is a silicone or derivative thereof having a molecular weight of from about 250 to about 40,000, preferably from about 500 to about 20,000, more preferably from about 1,000 to about 10,000 Da;
i₁ is an integer selected such that the monomer units constitute from about 30% to about 99.8% by weight of Polymer (i);
i₂ is an integer selected such that the monomer units constitute from about 0.1% to 50% by weight of Polymer (i);
i₃ is an integer selected such that the monomer units constitute from about 0.1% to 50% by weight of Polymer (i);
i₄ is an integer selected such that the monomer units constitute from 0% to 30% by weight of Polymer (i); and
i₅ is an integer selected such that the monomer units constitute from 0% to 20% by weight of Polymer (i);
(ii) polymers having the following formula: 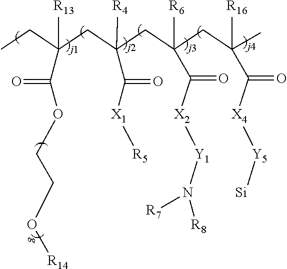
wherein
R₁₃, R₁₄, R₆, R₇, R₈ and R₁₆ are each independently selected from H or C₁-C₆ alkyl;
g is from 0 to 100, preferably g is 1;
X₁, X₂, and X₄ are each independently selected from O or NH;
R₅ is C₈-C₃₀ n- or iso-alkyl;
Y₁ and Y₅ are each independently selected from —(CH₂)ₘ—, wherein m is 1-30;
Si is a silicone or derivative thereof, preferably Si is at least one of a polydimethylsiloxane, an aminosilicone, a cationic silicone, a silicone polyether, a cyclic silicone, a fluorinated silicone and mixtures thereof; preferably Si is polydimethylsiloxane; preferably Si is a silicone or derivative thereof having a molecular weight of from about 250 to about 40,000, preferably from about 500 to about 20,000, more preferably from about 1,000 to about 10,000 Da;
j₁ is an integer selected such that the monomer units constitute from about 50% to 100% by weight of Polymer (ii);
j₂ is an integer selected such that the monomer units constitute from 0% to 50% by weight of Polymer (ii); and
j3 is an integer selected such that the monomer units constitute from 0% to 50% by weight of Polymer (ii); and
j4 is an integer selected such that the monomer units constitute from 0% to 50% by weight of Polymer (ii);
(iii) polymers having the following formula: 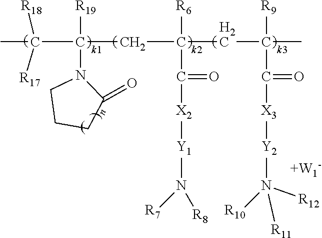
wherein
R₁₇, R₁₈, R₁₉, R₆, R₇, R₈, and R₉ are each independently selected from H or C₁-C₆ alkyl;
n is 0-4, preferably n is 1-4;
X₂ and X₃ are each independently selected from O or NH;
Y₁ and Y₂ are each independently selected from —(CH₂)ₘ—, wherein m is 1-30;
R₁₀, R₁₁, and R₁₂ are each independently selected from C1-C6 alkyl; preferably R10, R11, and R₁₂ are each independently selected from methyl, ethyl and propyl;
W₁— is a counter ion; preferably W₁— is selected from Cl—, Br—, I—, HSO₄⁻, CH3SO₄⁻, C2H5SO₄⁻, or OH—;
k₁ is an integer selected such that the monomer units constitute from about 50% to 100% by weight of Polymer (iii);
k₂ is an integer selected such that the monomer units constitute from 0% to about 50% by weight of Polymer (iii); and
k₃ is an integer selected such that the monomer units constitute from 0% to about 50% by weight of Polymer (iii); or
(iv)
polymers having a randomly substituted polysaccharide backbone comprising unsubstituted and substituted glucopyranose monomers and having a general structure according to Formula I: 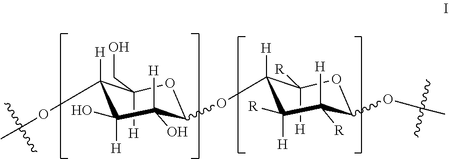
wherein each substituted glucopyranose monomer independently comprises from 1 to 3 R substituents, which may be the same or different on each substituted glucopyranose monomer, and
each R substituent is independently a substituent selected from the group consisting of hydroxyl, hydroxymethyl, R¹, R², R³ and a polysaccharide branch having a general structure according to Formula I; or a substituent selected from the group consisting of hydroxyl, hydroxymethyl, R¹, R², and a polysaccharide branch having a general structure according to Formula I, provided that at least one R substituent comprises at least one R₁ and at least one R²,
each R¹ is independently, the same or different, a first substituent group having a degree of substitution ranging from 0.01 to 0.2 and a structure according to Formula II: 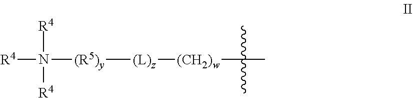
each R⁴ is a substituent selected from the group consisting of H; CH₃; linear or branched, saturated or unsaturated C₂-C₁₈ alkyl, provided that the sum of number of carbons of at least two of the R⁴ groups does not exceed 24, R⁵ is a linear or branched, saturated or unsaturated C₂-C₁₈ alkanediyl or a linear or branched, saturated or unsaturated secondary hydroxy(C₂-C₁₈)alkanediyl, L is a linking group selected from the group consisting of —O—, —C(O)O—, —NR⁹—, —C(O)NR⁹—, and —NR⁹C(O)NR⁹—, and R⁹ is H or C₁-C₆ alkyl, w has a value of 0 or 1, y has a value of 0 or 1, and z has a value of 0 or 1,
each R₂ is independently, the same or different, a second substituent group having a degree of substitution ranging from 0.001 to 0.5 and a structure according to Formula III: 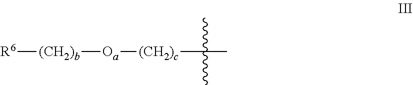
R₆ is a substituent selected from the group consisting of carboxylate, carboxymethyl, succinate, sulfate, sulfonate, arylsulfonate, phosphate, phosphonate, dicarboxylate, and polycarboxylate, a has a value of 0 or 1, b is an integer from 0 to 18, and c has a value of 0 or 1,
each R₃ is independently, the same or different, a third substituent group having a degree of substitution of 0 or ranging from 0.001 to 1.0, and having a structure according to Formula IV: 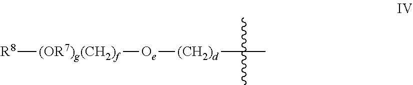
d has a value of 0 or 1, e has a value of 0 or 1, f is an integer from 0 to 8, g is an integer from 0 to 50, each R₇ is the group ethanediyl, 1,2-propanediyl, 1,2-butanediyl, or mixtures thereof, and R⁸ is an end group selected from the group consisting of hydrogen, C₁-C₂₀ alkyl, hydroxy, —OR¹ and —OR², and
the polymer has a weight average molecular weight ranging from 10,000 to 10,000,000 Daltons; or
(iv) mixtures thereof.

16. The personal care composition according to claim 15, where the polymer has a molecular weight of from about 200,000 to about 3,000,000.
